(19) United States
(12) Patent Application Publication
(10) Pub. No.: US 2012/0270209 A1
(43) Pub. Date: Oct. 25, 2012

(54) SYSTEMS, DEVICES, AND METHODS FOR SPECIFIC CAPTURE AND RELEASE OF BIOLOGICAL SAMPLE COMPONENTS

(75) Inventors: Ajay Shah, Cambridge, MA (US); Kenneth T. Kotz, Auburndale, MA (US); Mehmet Toner, Wellesley, MA (US)

(73) Assignees: MASSACHUSETTS INSTITUTE OF TECHNOLOGY, Cambridge, MA (US); THE GENERAL HOSPITAL CORPORATION, Boston, MA (US)

(21) Appl. No.: 13/320,732

(22) PCT Filed: May 14, 2010
(86) PCT No.: PCT/US2010/034943

§ 371 Date: Jun. 29, 2012

Related U.S. Application Data
(60) Provisional application No. 61/178,874, filed on May 15, 2009.

(51) Int. Cl. C12N 5/071; C12N 5/09; C12N 5/078; B05D 3/10; C12Q 1/68; C12M 1/00; G01N 21/62; G01N 33/53; C12N 5/0783

(52) U.S. Cl. 435/6.1; 435/7.1; 435/325; 435/283.1; 427/385.5

(57) ABSTRACT

Living cells can be selectively and reversibly bound to functionalized dissolvable material (e.g., cross-linked hydrogel compositions) and subsequently released from the composition as viable cells. In some examples, the cells are released by reducing the degree of cross-linking within a functionalized hydrogel composition and/or dissolving the functionalized hydrogel composition bound to the cells. The functionalized hydrogel compositions can be adhered to silicon- and silicon-oxide containing surfaces, such as glass and aminated silicon. The living cells can be isolated from biological samples, such as blood, by selectively binding certain cells from the sample to the functionalized hydrogel, removing unbound cells and later releasing viable bound cells from the functionalized hydrogel.

### CROSS-REFERENCE TO RELATED APPLICATIONS

This application claims the benefit of U.S. Provisional Patent Application Ser. No. 61/178,874, filed on May 15, 2009, the entire contents of which are hereby incorporated by reference.

### FIELD OF THE INVENTION

This disclosure relates to hydrogel coatings for selectively binding and releasing components, such as living cells, from biological samples.

### BACKGROUND

Isolation of specific cell populations from complex mixtures such as whole blood has significant utility in both clinical practice and basic medical research. A variety of approaches may be used to separate cells from a heterogeneous sample. For example, some techniques can use functionalized materials to capture cells based on cell surface markers that are particular to the target cell population using specific capture moieties present on or in the functionalized materials. Such capture moieties can include antibodies or other specific binding molecules, such as aptamers or selectins. For example, a microfluidic affinity-based chip that is configured to isolate rare circulating tumor cells (CTCs) from the whole blood of cancer patients is described, e.g., in Nagrath et al., “Isolation of rare circulating tumour cells in cancer patients by microchip technology,” Nature 450 (2007), pp. 1235-1239. These CTCs may disseminate from the tumor and are observed to be present in numbers that tend to correlate with patients' clinical courses. These CTCs may also be involved in metastasis. Accordingly, such microfluidic chip technology may be used in diagnostic and prognostic devices for oncological applications. At present, limited phenotyping and genotyping of these rare cells can be achieved because the CTCs tend to remain attached to the substrate (e.g., a silicon-based chip). The ability to release these cells would enable more detailed analysis of the CTCs, and aid in the understanding of the metastatic process.

A limitation common to many cellular capture techniques is the limited ability to recover captured cells following isolation. The ability to release cells following their specific capture would enable simple and direct nonoptical detection of the target cell population with much simpler methods and equipment. This capability of releasing specific captured cells may improve the accuracy of target detection, and can lower associated costs, processing time, and sample manipulation. Conventional techniques for releasing specifically captured cells include chemical methods, e.g., gradient elution, and mechanical approaches such as the use of bubbles within capillary systems. Such chemical and mechanical approaches can cause significant damage to the target cell populations; even if cell viability is preserved. For example, the ability to extract phenotypic and functional information from target populations may be compromised, because variations in chemical microenvironments and shear stress can cause significant changes in cellular expression patterns. In addition, some techniques rely upon the use of harsh chemistries—including very high or low pH environments—and/or significant variations in temperature or ionic strength that are not compatible with retention and release of viable cells from the surface.

Accordingly, there is a need for and interest in methods and materials which allow the release of specifically captured cells bound to a surface that is functional at a physiologic pH, ionic strength and temperature, and which do not exert undue chemical or mechanical stresses on the cells of interest.

### SUMMARY

This disclosure provides methods and surfaces for isolating components from a sample using functionalized hydrogel compositions, including the selective binding and subsequent release of cells from a blood sample. The invention is based in part on the discovery that living cells can be selectively and reversibly bound to certain functionalized hydrogel compositions while preserving cell viability. The functionalized hydrogel compositions can be adhered to a variety of surfaces and substrates, including silicon- and silicon-oxide containing surfaces, such as glass and aminated silicon. The living cells can be isolated from biological samples, such as blood, by selectively binding certain cells from the sample to the functionalized hydrogel, removing unbound cells and later releasing viable bound cells from the functionalized hydrogel.

In some embodiments, the substrate comprises a silica-containing material (e.g., glass, PDMS, sol-gel product or reactant). In some embodiments, the substrate could be polymeric thermoplastic materials including commodity or engineered polyolefin polymers or copolymers including but not limited to polyacrylics (Lucite, polymethylmethacrylate); polycarbonate (Lexan, Calibre, etc.); polyvinyl chloride, polyethylene, polypropylene, polyethylene terephthalate, cycloolefins (cycloolefin copolymer (COC, or TOPAS), or cycloolefin polymer (COP or Zeonor); polystyrene; epoxies, etc. In some embodiments, the substrate could be a thermosetting plastic, such as epoxies (mixture of epoxide resin with polyamine resin), including fiber-reinforced plastic materials. In some embodiments, the substrate could be any of these polymeric materials functionalized with silica. In some embodiments, the substrate could be metallic (gold, silver, platinum, copper, aluminum), metal oxides (copper oxide, aluminum oxide, silver oxide, indium tin oxide, etc.); inorganic materials including semiconductor materials and magnetic materials. In some embodiments, the substrate could be a combination of silica, polymeric, metallic, or inorganic listed above.

Methods for isolating and detecting living cells in a sample can include releasing a viable bound cell from a cell contact surface. For example, a method can include contacting a sample with a functionalized hydrogel comprising a cell-binding moiety bound to a cross-linked hydrogel polymer under conditions effective to bind the cell-binding moiety to a target cell from the sample, removing unbound cells from the sample, releasing the bound target cell from the functionalized hydrogel by converting at least a portion of the cross-linked hydrogel polymer to a non-cross-linked hydrogel polymer; and detecting the unbound target cell; wherein the unbound target cell is a viable cell. Such coatings or layers can be formed by applying an alginate gel onto a substrate or surface (e.g., using a spincoating process). The alginate can then be uniformly crosslinked using, for example, a calcium chloride spray. The crosslinked gel can be functionalized with a specific capture moiety such as, e.g., avidin. Such coatings can be dissolved to release captured cells using a dissolution agent such as, e.g., a solution containing a calcium chelator. In a further aspect, embodiments of the present invention include functionalized coatings or layers that are formed using acrylated alginate that is photocrosslinked. Such materials can be stable in the presence of anticoagulants that are calcium chelators, such as EDTA or sodium citrate, and can be dissolved to release captured cells using a material such as alginate lyase enzyme.

In some examples, the methods can include adhering a functionalized, cross-linked hydrogel layer on a functionalized surface using covalent bonds. In one example, a hydrogel layer up to about five micrometers thick can be covalently bound to a functionalized surface without requiring electrostatic attraction between the hydrogel and the surface. The surface can be functionalized by forming a layer of a binding moiety on the surface that is selected to covalently bind to either the hydrogel layer itself or to a primer material deposited between the hydrogel layer and the functionalized surface. Accordingly, the methods can include depositing a primer material onto a surface, depositing a hydrogel material onto the priming layer, cross-linking the hydrogel material on the primer material, and contacting the cross-linked hydrogel material with a functionalizing agent comprising a cell-binding moiety under conditions effective to bind the cell-binding moiety to the cross-linked hydrogel material, thereby forming the cell capture surface.

In a further aspect, this disclosure provides systems or devices that are capable of isolating specific cells from a biological sample (such as blood or another fluid), and then controllably releasing the captured cells without substantially affecting viability of the captured cells. Such systems and devices include one or more surfaces coated with a functionalized gel such as the alginate gels described above. Cell capture devices, such as biochips with functionalized surfaces, are described. Such cell capture devices can include, for example, the silicon CTC-chip described in Nagrath et al., “Isolation of rare circulating tumour cells in cancer patients by microchip technology,” Nature 450 (2007), pp. 1235-1239 and the herringbone device described in Int. Pat. App. Pub. No. WO 2010/036912(A2). The cell capture devices can include a primer material bound to a surface, a cross-linked functionalized hydrogel material chemically bound to the primer material, and a capture antibody. The primer material can include a polymercarbodiimide (e.g., 1-ethyl-3-(3-dimethylaminopropyl)-carbodiimide, or polysaccharide that is chemically bound to the surface; the hydrogel material can include a cross-linked polysaccharide which may be modified with other functional ligands such as, for example, biotin hydrazide. The hydrogel material can be foamed using a zero-length cross-linking process mediated by, for example, EDC and N-hydroxysulfosuccinimide (Sulfo-NHS). Preferably, the EDC is present in a molar ratio of at least about 1:20 relative to the monomers forming the cross-linked polysaccharide; and the capture antibody is chemically bound to the hydrogel material.

As used herein, the term “hydrogel” refers to a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels are also biocompatible, e.g., not toxic, to cells suspended in the hydrogel. The hydrogel can be a polysaccharide, such as alginate. The hydrogel can also cross-linkable molecules, such as one or more of the following: proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

As used herein, “functionalizing” a hydrogel material refers to chemical modification of the hydrogel material to modify the reactivity of the material. Similarly, functionalizing a surface refers to chemical modification of the surface to modify the reactivity of the surface. For example, the hydrogel material can be chemically modified by oxidizing, reducing, aminating or carboxylating one or more chemical functional groups. Functionalizing the surface can include, for example, contacting the surface (e.g., glass) with a chemical compound that introduces amine moieties to the surface. Functionalizing can be performed in one or more chemical reaction steps. A hydrogel can be functionalized by reactive contact with one or more functionalizing agents, which can be one or more chemical compounds that react with at least a portion of the hydrogel. For example, an alginate hydrogel can be functionalized by contact with a first functionalizing agent in solution (the first functionalizing agent comprising biotin hydrazide, a carbodiimide compounds and an amine compound) to form a functionalized alginate hydrogel, followed by surface binding of the functionalized hydrogel, cross-linking of the functionalized hydrogel bound to the surface, and contacting the cross-linked surface-bound functionalized hydrogel with a second functionalizing agent comprising streptavidin and then a third functionalizing agent comprising a biotinylated antibody. Preferably, a functionalized hydrogel material, can chemically bind a cell-binding moiety, such as an antibody or polynucleotide, that is selected to selectively bind a target in a biological sample (such as a living cell).

Unless otherwise defined, all technical and scientific terms used herein have the same meaning as commonly understood by one of ordinary skill in the art to which this invention belongs.

Although methods and materials similar or equivalent to those described herein can be used in the practice or testing of the present invention, useful methods and materials are described below. All publications, patent applications, patents, and other references mentioned herein are incorporated by reference in their entirety. In case of conflicting subject matter, the present specification, including definitions, will control. In addition, the materials, methods, and examples are illustrative only and not intended to be limiting.

These and other objects, features and advantages of the present invention will become apparent upon reading the following detailed description of embodiments of the invention, when taken in conjunction with the appended claims. The details of one or more embodiments of the invention are set forth in the accompanying drawings and the description below.

### BRIEF DESCRIPTION OF THE DRAWINGS

Further objects, features and advantages of the invention will become apparent from the following detailed description taken in conjunction with the accompanying figures showing illustrative embodiments, results and/or features of the exemplary embodiments of the present invention, in which:

FIGS. 1A-1D are schematic illustrations of a procedure for producing a functionalized hydrogel layer on a substrate in accordance with exemplary embodiments of the present invention;

FIG. 2 is a fluorescence image of a portion of a microfluidic device coated with an exemplary gel that has been labeled with a fluorescent marker;

FIG. 3 is plot of exemplary data relating thickness of a spin-coated alginate layer on a surface to spin speed;

FIG. 4 is a schematic illustration of a chemical process for functionalizing alginate using avidin as a capture moiety;

FIG. 5 is plot of exemplary data showing release behavior of alginate gel coatings;

FIG. 6 is plot of exemplary data showing functionalization efficiency of alginate gels using a bulk functionalization procedure;

FIG. 7 is plot of exemplary data showing dissolution behavior of alginate gel coatings using various chelating buffer solutions;

FIG. 8 shows exemplary fluorescence images showing dissolution of an exemplary gel that has been labeled with a fluorescent marker;

FIG. 9 is an exemplary fluorescence image showing a sealed channel in a device containing an alginate gel coating;

FIG. 10 is an exemplary bright field image showing CTCs and other cells that were captured and released from a patient blood sample using a functionalized gel layer;

FIG. 11 is plot of exemplary data showing a relationship between biofunctionality of alginate gel coatings and average density of biotins; and

FIG. 12 is plot of exemplary data showing acrylation efficiency of alginates that can be used to form functionalized coatings.

FIG. 13 presents a qualitative plot illustrating the relationship of dissolution vs. delamination as functions of shear stress.

While the present invention will now be described in detail with reference to the figures, it is done so in connection with the illustrative embodiments and is not limited by the particular embodiments illustrated in the figures.

### DETAILED DESCRIPTION

The present disclosure provides methods and materials for selective capture and release of viable cells, proteins, and the like, as well as to systems and devices that include such materials for selective capture and release. In one example, a coating or layer for specific cell capture is provided that includes a functional sacrificial hydrogel material. The functional coating can allow specific cell capture from biological samples such as, e.g., whole blood. Reducing the degree of cross-linking in the sacrificial layer (e.g., dissolving the functionalized hydrogel) can then release captured cells from the surfaces.

### Forming Cell Capture Surfaces

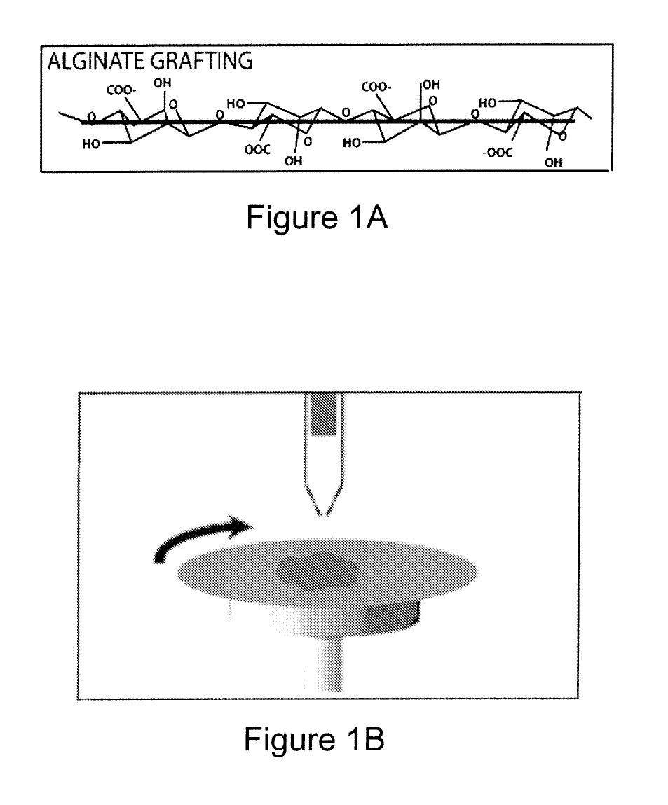

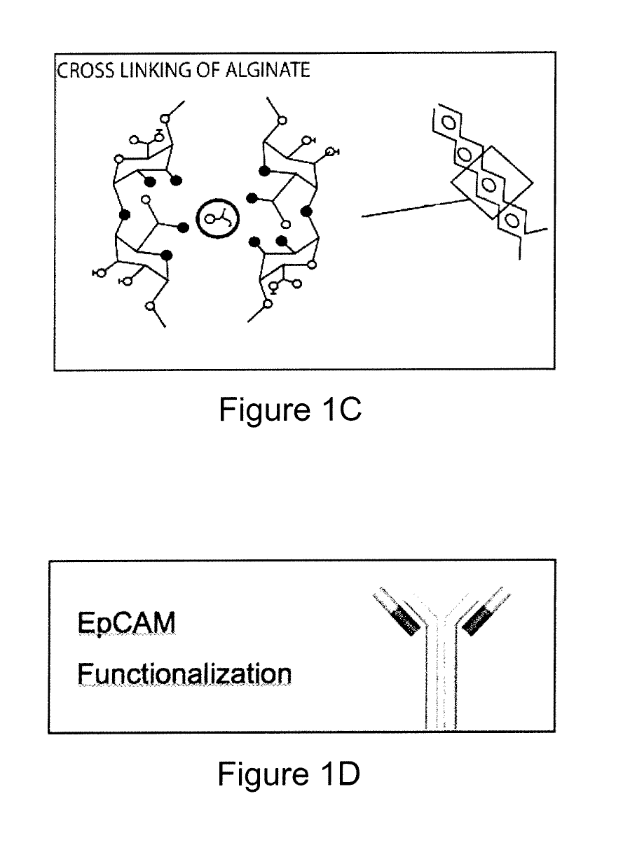

The cell capture surface can be formed by: (1) covalently adhering a hydrogel material onto a surface; (2) cross-linking the hydrogel material adhered to the surface; and (3) contacting the hydrogel material with a functionalizing agent comprising a cell-binding moiety under conditions effective to bind the cell-binding moiety to the cross-linked hydrogel material, thereby founing the cell capture surface. The hydrogel material can be contacted with the functionalizing agent before and/or after covalently adhering the hydrogel material onto the surface. In some examples, the hydrogel material is functionalized in solution prior to deposition onto a surface and prior to cross-linking of the hydrogel material bound to the surface (e.g., bulk functionalization). In other examples, the hydrogel material is deposited onto the surface, cross-linked and then contacted with a functionalizing agent to functionalize the hydrogel material. FIGS. 1A-1D illustrate an exemplary method of forming a cell capture surface.

Thin layers of hydrogel materials (e.g., less than about 10 micrometers thick, including layers having a thickness of about 5 micrometers or less) can be covalently adhered to surfaces. The hydrogel material can include one or more different polymers that can be cross-linked and attached to the surface. The surface can optionally be modified to include one or more chemical moieties selected to retain the hydrogel material, or to a primer material positioned between the hydrogel material and the surface. For example, the surface can be treated to introduce binding moieties selected to covalently bind to the primer material. In some examples, a carbohydrate hydrogel material can be covalently bound to a primer material containing a diimide compound, and the primer material can be bound to a surface having primary amine groups. Once bound to the primer material on the surface, the carbohydrate hydrogel can be cross-linked on the surface (e.g., using an ionic cross-linking agent or a photocrosslinking agent). The primer material can be deposited between the hydrogel material and the surface, for example by contacting a surface presenting suitable chemical functional groups with a solution of the primer material and a crosslinker, if needed. The primer material can be selected to form covalent bonds with both the hydrogel material and the functionalized surface to retain a hydrogel layer on the surface. The surface can be treated under conditions effective to introduce a chemical binding moiety capable of forming a covalent chemical bond with the primer material. In certain embodiments, a thin, substantially uniform coating of a hydrogel comprising alginate can be deposited on a glass substrate to form a cell capture surface that can be used for specific cell capture, such as a silicon chip configured to capture circulating tumor cells, or CTCs (a “CTC chip”).

In one example, a primer material including a carbohydrate such as alginate, shown in FIG. 1A, can be covalently bound to a functionalized glass surface, forming a grafted alginate primer layer covalently bound to the underlying glass surface. Prior to contact with the primer material, the glass surface can be treated to provide a functionalized surface having chemical moieties that covalently bind the primer material. For instance, the glass surface can be aminated by contacting a clean glass surface to a solution of an aminopropyltriethoxysilane, ethanol, and deionized water (e.g., having a pH of about 5) for suitable period of time (e.g., about five minutes) to aminate the glass surface. The aminated glass surface can be contacted with a solution of the primer material under conditions effective to covalently bind a layer of the primer material to the aminated glass surface. The primer material can be contacted with the functionalized glass surface as a solution containing a cross-linkable polysaccharide (e.g., alginate), a carbodiimide compound (e.g., 1-ethyl-3-(3-dimethylaminopropyl)-carbodiimide) with or without a succinimide compound (e.g., N-hydroxysulfosuccinimide, “Sulfo-NHS”) to stabilize the intermediate formed in the carbodiimide reaction. The functionalized glass surface can be immersed in a primer material solution at a pH of about 6.5. The primer material solution can include a molar excess of both the carbodiimide compound and the succinimide compound to the number of moles of uronic acid in the cross-linkable polysaccharide in the solution. The primer material solution can also include a molar excess of the carbodiimidecarbdiimide compound to the succinimide compound. For example, a primer material solution suitable for use in binding an alginate hydrogel layer to an aminated glass surface can include alginate functionalized using a process mediated by EDC and Sulfo-NHS in the solution with a molar ratio of 1 uronic acid:3430 EDC:1715 Sulfo-NHS, and with 1 mg/mL of alginate in a 50 mM MES buffer solution having pH of about 6.5.

The cross-linkable hydrogel material can be adhered to the surface by spin coating a solution of the hydrogel material onto a rotating surface. Alternatively, the cross-linkable hydrogel material can be adhered to the surface by other techniques, or combinations of techniques, including drop-deposition and/or spray deposition. Optionally, the hydrogel material can be functionalized in solution to bind to a cell-binding moiety, prior to deposition onto the surface. The rotating surface can include a surface layer of primer material covalently bound to an underlying surface, such as the alginate-containing primer material adhered to a glass surface described above. A thin layer (e.g., less than 10 micrometers thick) of a cross-linkable hydrogel material attached to a glass surface can be formed by spin coating a solution of the cross-linkable hydrogel material onto a rotating surface of the priming material covalently bound to an underlying functionalize glass surface. For example, as shown in FIG. 1B, a viscous 2% alginate solution in deionized water can be dispensed onto a substrate (e.g., a glass slide or a CTC chip) until it is substantially covered. The substrate can then be spun at a speed selected to provide a substantially uniform coating layer while removing excess solution. For example, the solution may be spun on the substrate for about 30 seconds, or for about one minute. The coating solution can then be dried to form a film on the substrate.

In some examples, the cross-linkable hydrogel material comprises a cross-linkable carbohydrate such as the polysaccharide alginate. Alginate is a naturally-derived biomaterial isolated from brown algae that exhibits a number of favorable properties in biotechnology applications. Alginate is a cytocompatible, non-fouling biomaterial that is generally regarded as safe by the U.S. Food and Drug Administration. Standard grade alginate (A2033) can be obtained, e.g., from Sigma-Aldrich (St. Louis, Mo.), and fluorescent beads (G50) used to assess dissolution of gel coatings can be obtained, e.g., from Duke Scientific (Palo Alto, Calif.). Alginate is a linear polysaccharide having a backbone of repeating mannuronic and guluronic acid monomers. Each monomer contains a readily functionalizable carboxylic acid, which can be readily functionalized to enable specific cell capture as described herein. Alginate can form temperature independent gels via divalent cation crosslinking (using, e.g., calcium cations) under physiologic conditions. The gelation of alginate can be reversed by processes such as, e.g., chelation of a crosslinking cation.

FIG. 1B illustrates an exemplary spin coating process that can be used to coat the substrate with a cross-linkable hydrogel solution containing alginate. Optionally, the hydrogel solution can include a functionalized alginate adapted to bind to a cell-binding moiety. The presence of the primer layer between an alginate-containing hydrogel layer and a glass surface can improve adhesion and mechanical stability of a subsequently applied coating. Stability of exemplary gel coatings containing alginate can be improved by grafting an alginate priming layer to a glass substrate surface prior to coating the surface with the alginate hydrogel solution. A covalently grafted priming layer may be anchored to the surface as shown, e.g., in FIG. 1A, and the associated alginate chains may be capable of interpenetrating with alginate chains present in the subsequently applied gel coating. Such grafted glass slides were observed to be very hydrophilic, and exhibited contact angles of less than about 10°. In contrast, control slides in which 1-Ethyl-3-(3-dimethylaminopropyl)-carbodiimide (EDC) was omitted from the grafting reaction and aminosilinated slides exhibited contact angles greater than 30°. Gels formed on the exemplary grafted substrate surfaces were observed to be mechanically stable for over 48 hours when immersed in 1 mM calcium chloride in TBS, as shown in Table 1.

TABLE 1
Observed Stability of Gel Coatings on Various Treated Surfaces

| Surface Treatment | Time to failure in |
|---|---|
| (n ≧ 3) | 1 mM CaCl₂ in TBS |
| Piranha cleaned slide | 1.5 min |
| Plasma treated | <15 min |
| (50 W for 35 sec) | |
| Plasma treated | <15 min |
| (200 W for 2 min) | |
| Amine functionalized | 100 min |
| Poly-1-lysine treated | <70 min |
| Succinic anhydride | <70 min |
| (carboxyl) functionalized | |
| Avidin functionalized | <1 min |
| Epoxy functionalized + UV | 20 min |
| Surface Grafted Alginate | All samples stable at |
| (n = 5) | 48 Hours |

Mechanical stability observations for exemplary alginate hydrogel coatings (based on time to failure of the coatings) are shown in Table 1 for a number of different surface treatments. None of the direct ionic, non-covalent or covalent surface modifications tested was observed to stabilize a bare glass gel-substrate interface for more than about 100 minutes without a primer material, and many gel coatings had much shorter lifetimes until failure. Based on factors such as fast diffusion of ions in an aqueous solution and the short length scales involved (e.g., sub-micron thickness of the coated gel layers), Gopferich theory suggests that the gels may be bulk eroding, and that mechanical failure may result from an interfacial failure between the gel and the surface due to competing ionic strengths. However, such alginate hydrogel coatings were observed to be stable for over 24 hours when immersed in deionized water when the alginate hydrogel coatings were deposited on an alginate-containing primer material covalently bound to an aminated glass surface (“surface grafted alginate”).

Referring to the data shown in FIG. 3, the thickness of spin coated surface grafted hydrogel material were evaluated by applying an alginate solution to a substrate using different spin speeds and measuring the resulting thickness of the hydrated gel. Surface grafted hydrated alginate hydrogel thickness and surface roughness were measured using a non-contact confocal microscope with materials characterization software (Olympus LEXT OLS3). Exemplary results of this procedure are presented in FIG. 3. Each data point shown in FIG. 3 represents an average of three or more independent measurements, and each error bar represents the standard error of the mean. A substantially linear correlation was observed between gel thickness and spin speed (with an r² correlation coefficient of 0.94), which is in general agreement with spin coating theory. It was also observed that the variation in coating thickness generally decreased with increasing spin speed. Further, these exemplary gel coatings or film had an average surface roughness of about 37+23 nm (RMS value). Based on these results, for example, gel films having a thickness of just under a micron can be formed by spinning the applied films at about 3000 RPM.

After covalently adhering a cross-linkable hydrogel material, such as alginate, with a desired thickness (e.g., less than about 10 micrometers) onto the surface, the hydrogel material can be cross-linked while attached to the surface (e.g., the surface grafted alginate described above). Preferably, substantially all of the dried film is crosslinked in one procedural step to prevent the film from folding up onto itself, tearing, or otherwise destabilizing. FIG. 1C shows a cross-linking procedure that can be performed to cross-link the hydrogel solution coating deposited in FIG. 1B. In the example shown in FIG. 1C, a hydrogel solution comprising alginate is crosslinked by contacting the alginate bound to a primer layer on a surface with a cross-linking agent, such as a calcium chloride solution. Such crosslinking can be achieved with aerosolized particles of calcium chloride, e.g., using an airbrush (H-Set from Paasche Corp., Chicago, Ill.) to spray the dried films with a 250 mM solution of calcium chloride in a Tris Buffered Saline (TBS). For example, the spray solution can include 25 mM TBS and 150 mM NaCl, with a pH of about 7.2. The brush spray pressure and distance from the sample can be selected to produce uniform droplets having a size on the order of a micron. For example, using an air brush pressure of about 80 PSI and spraying the substrate from a distance of about 8 inches can produce such micron-sized droplets. This spray technique can be used to rapidly and uniformly crosslink the films to form a coating of stable hydrogel. This exemplary technique for uniformly coating a substrate with a crosslinked alginate layer was evaluated by mixing 50 nm fluorescent beads into the initial alginate solution at a concentration of 0.03% (wt/vol) prior to spin coating. The samples were then crosslinked, trapping the beads within the gels that were formed. After multiple washes to remove any loose beads, the gels were then imaged.

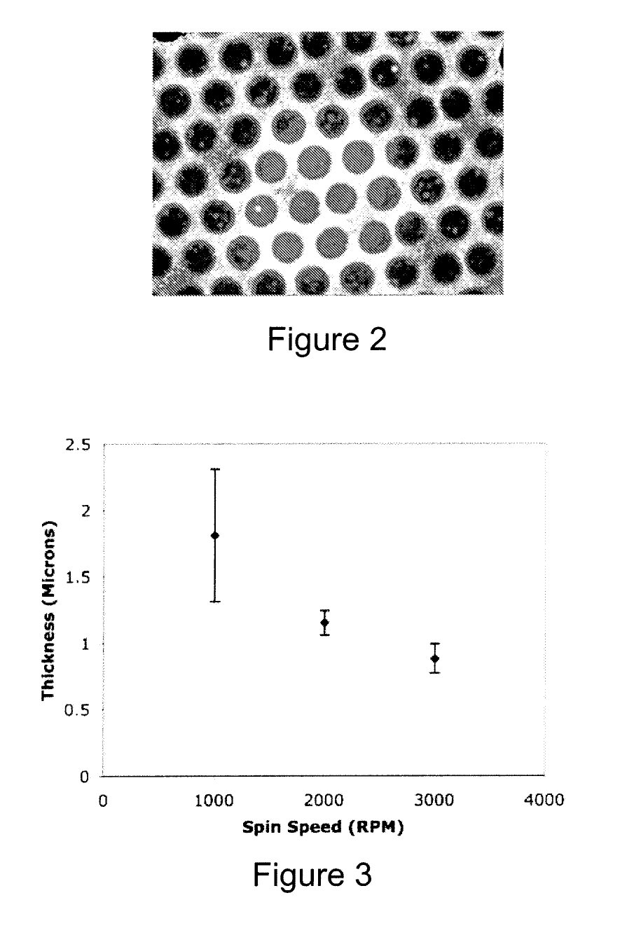

A fluorescent image of a cross-linked alginate coating attached to the surface of a microfluidic device (e.g., a CTC chip) is shown in FIG. 2. To faun crosslinked hydrogels, the films were spray-crosslinked with a solution of 250 mM calcium chloride in TBS using an airbrush at 80 PSI pressure from a distance of about 8 inches. The uniform fluorescence observed in this image suggests that the surfaces of the CTC chip are covered uniformly, with no significant bare spots. The dark circles in FIG. 2 represent the tops of circular posts that protrude from the base of the exemplary microfluidic device, a CTC chip, used to evaluate coating uniformity. The spacing between the posts can be as narrow as about 30 microns in some locations. Thus, it is generally preferable that the applied layer of hydrogel or other coating does not significantly narrow or restrict this gap. A significant and/or non-uniform narrowing of such gaps may change the fluid flow profile through the channels and could negatively affect cell capture or other performance of the device. For example, the thickness of the gel layer in certain embodiments is preferably less than about 2 microns, or more preferably less than about 1 micron. Lager thicknesses may be used in other embodiments, e.g., for coating substrates having features with larger dimensions than the microfluidics channels containing the surface-bound layer of hydrogel material.

The hydrogel material can be functionalized before and/or after deposition and cross-linking of the hydrogel material on the surface. For example, the hydrogel material can be deposited and bound to a primer material on a surface, cross-linked on the surface and then contacted with a functionalizing agent that chemically binds to the cross-linked hydrogel material. In another example, the hydrogel material is first functionalized in solution, then deposited and bound to a primer material on a surface, and then cross-linked. In other examples, a functionalized hydrogel material is formed in solution, deposited on and bound to a primer material on a surface, cross-linked on the surface and then contacted with a functionalizing agent. The hydrogel can be functionalized through contact with multiple functionalizing agents. For example, the hydrogel can be contacted with a first functionalizing agent in solution, prior to deposition onto a primer material, deposited and cross-linked on the primer material to form a cross-linked functionalized hydrogel, and then contacted with a second functionalizing agent to bind a cell-binding moiety to the functionalized cross-linked hydrogel.

In a first aspect, a cross-linked hydrogel material is functionalized after deposition onto a surface, for example by contacting the cross-linked hydrogel material with a functionalizing agent. To form the cell capture surface, the cross-linked hydrogel material bound to a surface (e.g., as described above) can be functionalized by contacting the cross-linked hydrogel material with a functionalizing agent comprising a cell-binding moiety under conditions effective to bind the cell-binding moiety to the cross-linked hydrogel material, thereby forming the cell capture surface. In some examples, the cross-linked hydrogel material contains cross-linked alginate. Alginate presents a single carboxyl group per monomer. Conventional carbodiimide chemistry techniques can be used to modify the carboxyl group. Such chemistry techniques can provide a number of further advantages including, e.g., allowing a robust one-step process that may not require protecting/de-protecting of the treated surfaces, and reducing a likelihood of self-crosslinking because alginate has free carboxyl groups, whereas proteins and/or antibodies of interest may include free amines and not have such carboxyl groups. Preferably, the hydrogel material is a thin film of material (e.g., less than about 10 micrometers thick) that is first covalently bound to the surface (e.g., through a primer material positioned between the hydrogel material and the functionalized surface), and then cross-linked while covalently bound to the surface (e.g., by contacting the bound layer of hydrogel material with a cross-linking agent). The cross-linked hydrogel layer can be functionalized to include a cell binding moiety, such as an antibody (FIG. 1D).

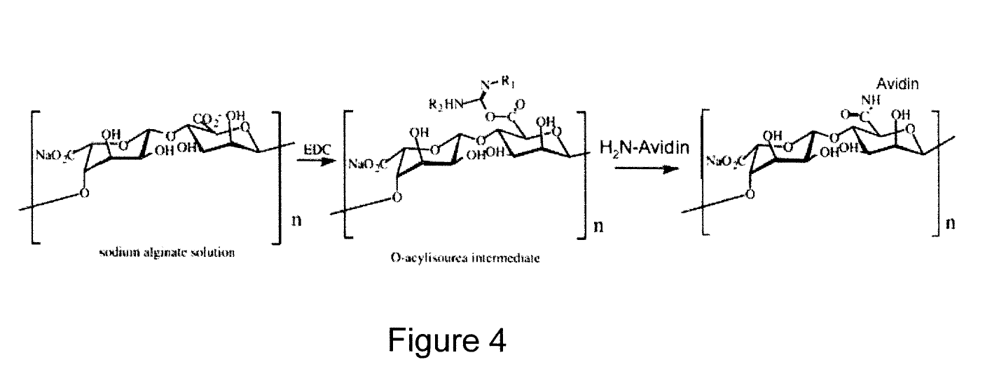

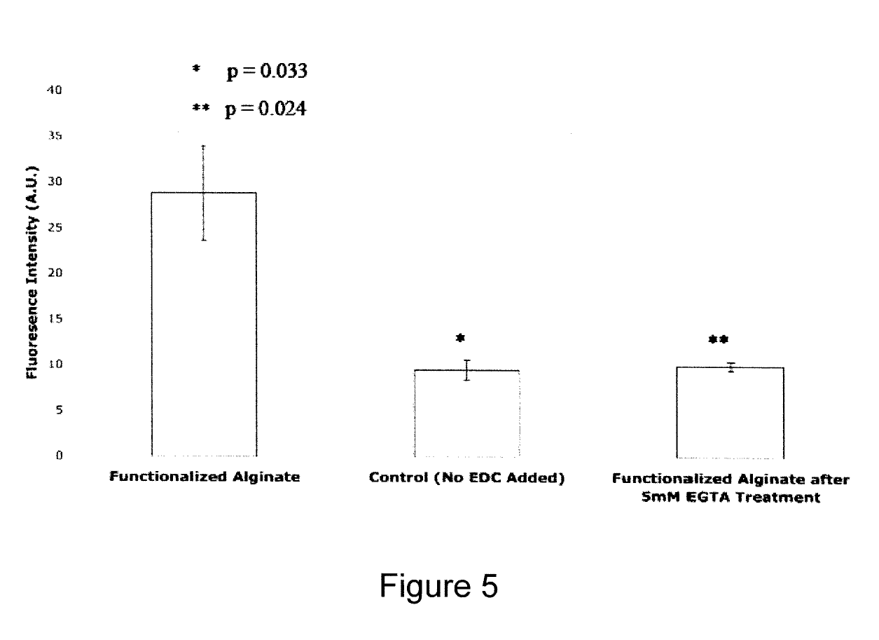

For example, carbodiimide chemistry can be used to link avidin to a cross-linked hydrogel surface, as shown in FIG. 4. In an exemplary functionalization procedure, 1-Ethyl-3-(3-dimethylaminopropyl)-carbodiimide (EDC) was used at a 1:20 molar ratio relative to the number of free uronic acid groups on the alginate, and N-hydroxysulfosuccinimide (Sulfo-NHS) was provided in the solution at a 2:1 ratio relative to the EDC. Avidin was also included in the solution at a concentration of 10 μg/mL. The crosslinked gel was exposed to this solution, and the unbuffered reaction was allowed to proceed for about 3 hours, followed by a 45 minute wash. The functionalization achieved using this exemplary procedure was assessed by comparing the fluorescent intensity of avidin treated with fluorescein isothiocyanate (FITC), e.g., FITC-avidin, coupled to a gel using EDC, as described herein, to a control sample in which EDC was omitted. Exemplary fluorescence measurements are shown in FIG. 5. Each data point shown in FIG. 5 represents an average of three or more independent measurements, and each error bar represents the standard error of the mean. Statistical significance was determined by calculating p values using a two-tailed students' t-test assuming unequal variances. The higher fluorescence intensity observed in the functionalized alginate coatings or layers, as compared to that of a control coating that had no EDC added, indicates the presence of specific carbodiimide-mediated functionalization.

In a second aspect, the hydrogel can be functionalized in solution prior to surface deposition and cross-linking. Hydrogel materials can be functionalized before deposition on the surface by using a bulk functionalization technique where a hydrogel is reacted in solution prior to deposition of the hydrogel onto a substrate or primer material, and prior to cross-linking. For example, hydrogel functionalization can be achieved by introducing or adding certain materials to the gel solution (e.g., an alginate solution) to functionalize or otherwise chemically modify the hydrogel, coating the resulting modified hydrogel solution onto a surface or primer material substrate (e.g., spin-coating), and then cross-linking the deposited modified hydrogel solution. Such bulk techniques can provide a highly repeatable and scalable functionalization of coating materials. For example, the alginate can be modified with avidin in solution. In one exemplary procedure, biotin was coupled to the alginate backbone and gels were then formed using the biotin-alginate material. This procedure was optimized by varying the coupling of biotin to the alginate and then incubating the biotinylated gels with FITC-avidin to determine the amount of biotinylation that would saturate the surface of the gel with avidin. The functionalized hydrogel can include chemical moieties selected to covalently bind to cell-binding moiety after surface deposition and cross-linking of the functionalized hydrogel.

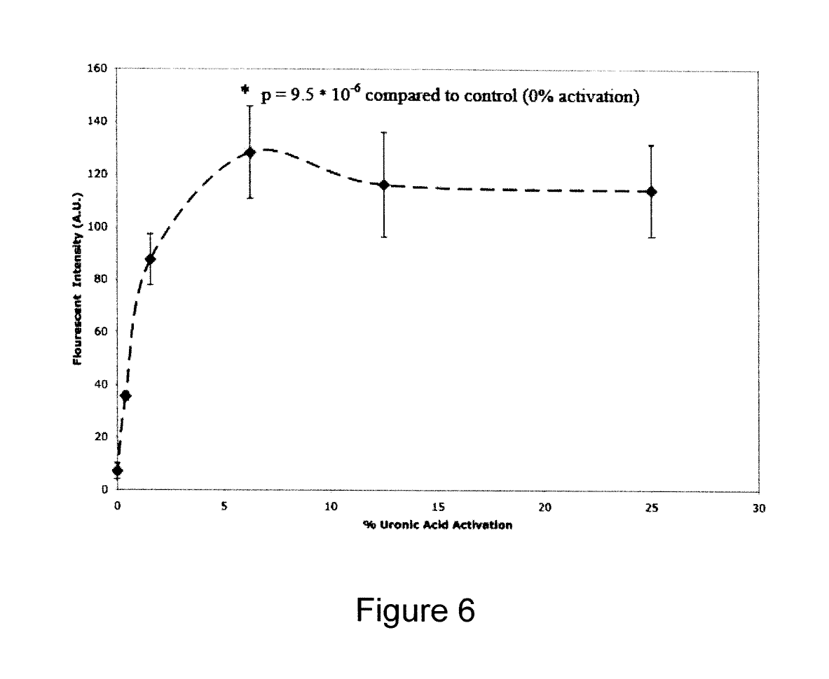

In another example of the bulk functionalization procedure, biotin-hydrazide was used to modify the alginate in a solution prior to deposition onto a primer material substrate. Biotin hydrazide was mixed with a 1% (w/v) alginate solution at a 1:2 molar ratio relative to the free acid groups. EDC and N-hydroxysuccinimide (NHS) were used in a 1:2 molar ratio, and the EDC:free acid ratio was varied (results shown in FIG. 6). Following a three hour reaction in a 50 mM MES solution having a pH of about 6.5, the alginate was dialyzed (with a molecular weight cutoff at 10,000) for about 72 hours to remove any unbound biotin hydrazide and EDC, and then lyophilized. To determine a preferable EDC:alginate ratio, the EDC concentration was varied and the lyophilized materials were reconstituted to 2% concentration in deionized water. This solution was spun-coated onto glass slides, crosslinked, and incubated with FITC-avidin for about 30 minutes. The bulk functionalization chemistry was quantitatively assessed by capturing pre-labeled H1650 cells on the functionalized gels, and the results compared to capture using an unfunctionalized control. As shown in FIG. 6, uronic acid activation was varied by varying the EDC concentration while maintaining an excess amount of biotin hydrazide during bulk functionalization. Each data point shown in FIG. 6 represents an average of three or more independent measurements, and each error bar represents the standard error of the mean. Avidin saturation on gels formed with these materials was observed at 6.25% activation. To ensure saturation in the coatings, and avoid potential variability in the chemistry, such functionalized materials can be prepared at an activation of about 10%.

Further, alginate chain length and/or polydispersity can affect functionalization of the alginate during bulk functionalization. For example, using longer alginate chains (e.g., those having an average MW of about 220 kD) can produce higher levels of biofunctionality than when using shorter alginate chains (e.g., those having an average MW of about 100 kD). However, the alginate hydrogels formed using chains having a MW of about 100 kD exhibited a wider polydispersity, which may lead to a broader range of biotinylation. Accordingly, a higher degree of polydispersity in the alginate chains may reduce the resulting biofunctionality, and it may be preferable to produce functional gels using alginate chains that are less polydisperse.

In another example, a cross-linkable hydrogel coating was formed on an aminated glass CTC chip surface by depositing a biotinylated alginate solution onto the alginate-containing primer material described above. The biotinylated alginate solution can be prepared by combining a 1% (w/v) solution of alginate (100 kD) in 50 mM MES buffer solution having a pH of about 6.5 with biotin hydrazide (at a molar ratio of 1 uronic acid:0.2 biotin hydrazide), EDC and Sulfo-NHS (molar ratio: 1 uronic acid: 0.1 EDC: 0.05 Sulfo-NHS). This solution can be lyophilized and reconstituted at a 2% concentration in deionized water. The reconstituted aqueous 2% biotin-alginate solution can be deposited onto and covalently bound to the grafted alginate primer material substrate (described above) at 3000 RPM for 30 seconds and then air dried.

A cell-binding moiety can be incorporated in a functionalized hydrogel by contacting the functionalized cross-linked hydrogels with a solution of the desired cell-binding moiety. The functionalized cross-linked hydrogels can be formed by various methods, including the bulk-functionalization method (i.e., functionalizing the hydrogel prior to deposition and/or cross-linking of the hydrogel) and/or by contacting a cross-linked hydrogel bound to a surface with a functionalizing agent. For example, the cell-binding moiety can be a biotinylated EpCAM antibody that can be contacted with a cross-linked functionalized hydrogel adapted to covalently bind the biotinylated antibody. Functionalized hydrogel materials allow specific cell capture by a surface-bound layer of the functionalized hydrogel material. The functionalized hydrogel can include, for example, antibody cell-binding moieties bound to biotinylated alginate.

Alternatively, the cell capture surface can be formed by covalently adhering other cross-linkable hydrogel materials to a surface (e.g., with or without a primer material). In another example, a cross-linkable hydrogel material containing polyethylene glycol (PEG) can be adhered to a surface by first functionalizing the surface with an acrylate moiety, and then covalently binding a diacrylate PEG derivative in a primer material to the functionalized surface. The cross-linkable hydrogel material adhered to the surface can then be cross-linked while bound to the surface. The cross-linked hydrogel material bound to the surface can then be contacted with a functionalizing agent comprising a cell-binding moiety under conditions effective to bind the cell-binding moiety to the cross-linked hydrogel material, thereby forming the cell capture surface.

In other examples, the hydrogel can be photocrosslinked. The hydrogel can be biotinylated for functionalization. For example, acrylation of an alginate hydrogel can be achieved by reacting the hydroxyl group on the alginate with an excess of methylacrylic anhydride, leaving carboxyl groups available for biotinylation. Exemplary data showing the degree of alginate acrylization as a function of excess methacrylic anhydride used are shown in FIG. 12. Photocrosslinked alginate gels that are stable for over 7 days in EDTA solutions can be formed using such acrylated alginate and introduction of a photoinitiator.

In another approach, an acryl modified alginate for photocrosslinking can be formed using water soluble approach that is based on using N-(3-Aminopropyl)methacrylamide HCl. As this is a methacryl-containing molecule with an amine on one end, it can be conjugated to the alginate via the same carbodiimide chemistry used to attach the biotin hydrazide. Thus, a single conjugation reaction can be used to form an alginate polymer with a backbone that is ‘decorated’ with multiple ligands (e.g., biotin and methacryls) at the desired stoichiometric ratios. We have made algiantes with a wide range of biotin densities (0-20%) and acryl densities (0%-75%), where the % refers to the starting ratio of the ligand to the number of free carboxyl groups on the alginates in solution. Furthermore, when up to 75% of the total number of available carboxyls are targeted, the efficiencies appear to remain constant at approximately 55% for any combination of either ligand. The resulting material retains its ability to be calcium crosslinked, may also be photocrosslinked in the presence of a photoinitiator e.g., Irgacure 2959, and may be functionalized with avidin. This approach may be extended to include any number of small molecule ligands containing primary amines and no carboxyls. The protocol is identical to that on line 10, page 24 except that both ligands are mixed in.

In one exemplary procedure, stable photocrosslinked alginate gels that remain gelled in the presence of calcium chelators can be formed by spin coating acryl alginate onto a surface or substrate. The gel layer can then be sprayed with a solution of calcium chloride (at a concentration of about 100-250 mM or higher). The application of the calcium chloride solution rapidly forms calcium crosslinked alginate gels. These gels can then be incubated in a calcium-containing solution that includes a photoinitiator at appropriate concentrations (e.g., Irgacure 1959 photoinitiator at a concentration of about 0.05-0.5%). After incubation, the gels can be treated with UV light (e.g., for a duration between about 30 seconds and about 10 minutes) to initiate free radical polymerization of the alginate.

In a further exemplary procedure, such stable alginate coatings can be produced by mixing a photoinitiator with acryl alginate prior to spin coating the acryl alginate onto a surface or substrate. The dried substrate can then be exposed to UV light to crosslink the films. Such films or coatings formed from acrylated alginate can produce stable gels when hydrated without using a calcium crosslinking process. Acrylated alginate hydrogels are useful, for example, to provide a material that can be used with blood samples that have been treated with a calcium chelator (e.g., EDTA or sodium citrate).

### Hydrogel Formulations for Cellular Release

Once formed, cell capture surfaces can be contacted with multi-component biological samples (e.g., blood) to selectively capture and retain components from the biological sample (e.g., living cells) at the binding moiety (e.g., an antibody) attached to the surface-bound functionalized hydrogel. The captured material, such as cells from the biological sample, can be released from the cell capture surface by reducing the amount of cross-linking in the functionalized hydrogel that is bound to the binding moiety. The functionalized hydrogel layer adhered to the surface and/or primer material can act as a sacrificial hydrogel layer that dissolve when the degree of cross-linking in the functionalized hydrogel layer is sufficiently reduced, leading to release the material bound to the binding moiety. For example, a cell-binding moiety bound to a captured viable cell can be released from a functionalized hydrogel comprising alginate with an agent that reduces the cross-linking in the alginate.

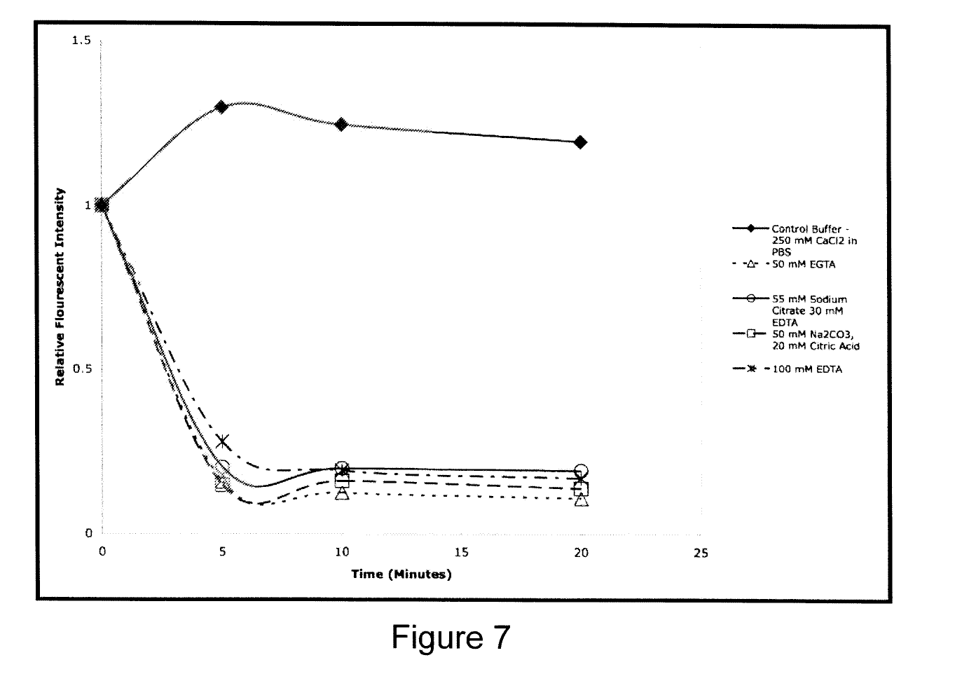

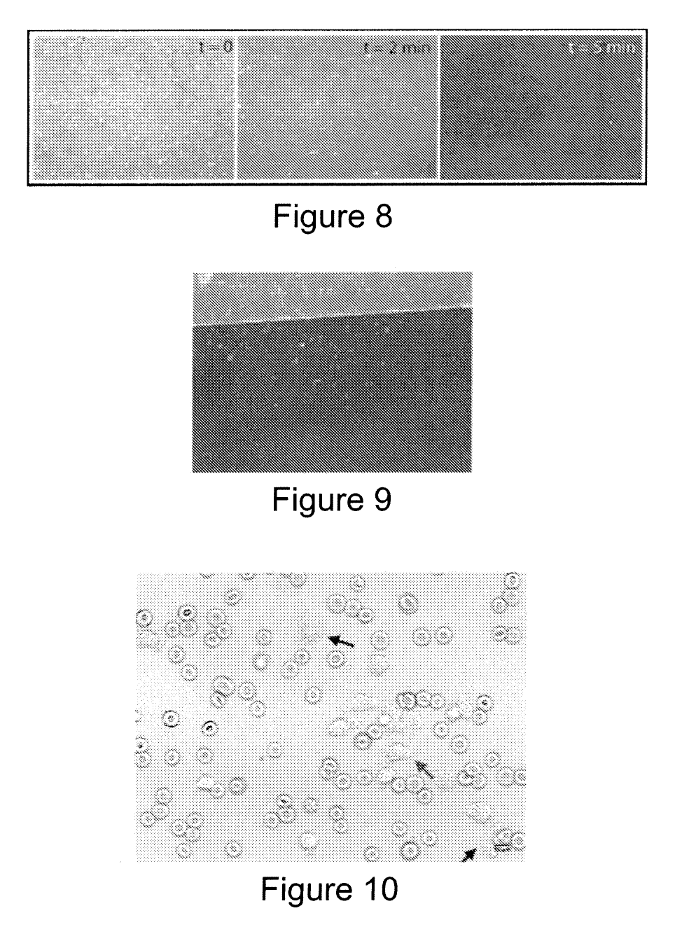

In one example, the sacrificial cross-linked functionalized hydrogel can include cross-linked calcium-alginate (e.g., as described above), an ionic cross-linked hydrogel material, that is cross-linked with a calcium ion to form the hydrogel and subsequently dissolved by contact with a calcium chelating agent. Various chelating buffers were evaluated for their ability to dissolve such exemplary gel coatings. Dissolution was measured by impregnating fluorescent beads in prepared gel coatings, and then measuring the decrease in fluorescence as the beads were released from the dissolving gel upon exposure to the various buffers. Several calcium chelating agents were evaluated in this manner, including: 50 mM EGTA in RPMI 1640 medium, 55 mM Sodium Citrate with 150 mM Sodium Chloride and 30 mM EDTA, 50 mM Sodium Carbonate with 20 mM Citric Acid, and 100 mM EDTA in PBS. A solution of 250 mM Calcium Chloride in PBS was used as a control to account for any change in fluorescence based on time or exposure to a non-chelating solution. All of these chemicals can be obtained, e.g., from Sigma-Aldrich. The fluorescence of the gel coatings was measured before and during chelation treatment (e.g., at exposure times of 5, 10, and 20 min). Exemplary results of these fluorescence measurements are shown in FIG. 7. Based on these results, a solution of EGTA in RPMI was observed to be a preferred chelating buffer because it exhibited the most rapid dissolution. This chelating buffer also has an appropriate pH and ionic strength for maintaining cell viability. To reduce potential effects on cell viability arising from exposure to the chelating agent, the EGTA concentration in the buffer solution was lowered to 5 mM. Dissolution of exemplary gel coatings exposed to a flow of this more dilute buffer solution (at shear stresses comparable to those often used in conventional CTC chips) was observed. FIG. 8 shows three fluorescence images of such a surface exposed to this dilute buffer solution. The increasing darkness of the images from t=0 to t=5 min indicates a progressive dissolution of the gel coating containing fluorescent beads. For example, the observed fluorescent signal was observed to decrease to about 30% of the initial signal after an exposure time of 5 minutes, which is comparable to the results shown in FIG. 7. Further observations of alginate-calcium ion cross-linked hydrogel dissolution using this exemplary buffer solution containing 5 mM EGTA are shown in FIG. 5. The rightmost bar in this graph indicates that fluorescence levels of the functional gel surface after exposure to the EGTA buffer solution comparable to levels observed in the control sample (that contained no fluorescent beads). These observations suggest that exposure to the 5 mM EGTA buffer solution removed substantially all of the functionalized gel coating.

The calcium-alginate functionalized hydrogel system described herein can be used for a variety of biological sample applications. It may be preferable for processing samples of heparinized blood, as heparin does not tend to affect the native calcium concentration of blood. However, it may be desirable to process samples of blood that has been treated with calcium chelators such as EDTA or sodium citrate, which are common anticoagulants. Such calcium chelators tend to dissolve the calcium-alginate materials described herein. However, such calcium chelating anticoagulants can be desirable for use in affinity-based cell capture systems, because they can decrease non-specific binding as compared to heparinized blood. This benefit derives from calcium being a signaling molecule for cell adhesion, such that its chelation can significantly limit cell attachment to a surface, and thereby increase purity of specifically captured cells.

In another example, the functionalized hydrogel material can be dissolved using an enzyme such as a lyase. For example, a functionalized hydrogel material bound to a cell-binding moiety that is at least partially photocrosslinked can be dissolved by contact with a lyase enzyme in solution. Acrylated alginate can be crosslinked to form covalent gels using a photoinitiator and UV light. Photocrosslinked gels of acrylated alginate as described herein can be degraded using substances such as, e.g., alginate lyase, a bacterial enzyme that interacts with the alginate backbone. Such specific interaction can be important, because rapid gel degradation is preferable for releasing cells captured by the functionalized coatings or layers.

Degradation of such photocrosslinked gels of acrylated alginate by lyase can be achieved in a reasonable timeframe (e.g., less than about 5 minutes) if the crosslinking density is well-controlled. For example, acryl alginate gels that are photo-crosslinked in baths of a 250 mM calcium chloride solution may not be reliably digested in shorter times. However, such gels that are photo-crosslinked in solutions of 2.5 mM calcium chloride can be more rapidly digested.

This difference in behavior may be related to the density of covalent crosslinking. For example, cross-linking in a 250 mM calcium chloride solution may pull the alginate chains closer together, enabling a free-radical propagation to reach more chains in a given path as compared to a configuration having chains that are further apart.

In another example, an acrylated alginate hydrogel can be cross-linked by two or more different methods. It may be preferable to initially crosslink such gels using, e.g., a 250 mM calcium chloride solution to promote rapid initial gelation. Accordingly, a procedure may be used in which the gels are initially crosslinked in a 250 mM calcium chloride solution, and then placed in successive baths of 2.5 mM calcium chloride to ‘wash out’ excess calcium. Gel coatings formed using this exemplary procedure can remain stably crosslinked at 2.5 mM, and can then be photocrosslinked. The subsequent photocrosslinking can forms a lower crosslink density, because the chains may be relaxed in the lower concentration calcium bath. Such photocrosslinked gels can be more easily degraded by the addition of alginate lyase.

Forming the functionalized hydrogel material can also include enzymatically digesting the alginate (using, e.g., 1 mg/mL alginate lyase in PBS for 1 hour) prior to conducting the assay. Performing this digestion for as little as 20 minutes can be sufficient to significantly reduce the alginate chain length, thereby enabling greater accuracy and repeatability when applying HABA absorbance assays.

### Microfluidic Devices Including a Functionalized Hydrogel Material

The functionalized hydrogel materials can be included in microfluidic devices to capture and then release living viable cells from the hydrogel material. To evaluate the functionality of exemplary coating materials described herein, specific capture and release of cells under flow conditions was performed. Functional gel coatings were formed, crosslinked, and dried as described herein. Exemplary elastomer microchannels were fabricated and clamped on top of these films. Such microchannels are described, e.g., in Cheng et al., “A microfluidic device for practical label-free CD4+ T cell counting of HIV-infected subjects.” Lab on a Chip 7 (2007), pp. 170-178.

To ensure that the clamped system of microchannels and a coated surface was sealed properly, fluorescent beads were impregnated in some gels used to coat the surface and EGTA was flowed through these systems. The exemplary image shown in FIG. 9 includes a darker region within the microchannel and a lighter area outside the microchannel, with a sharp line separating the two areas. These results suggest that the liquid seal was secure, with the portion of the gel inside the microchannel wall dissolved (leading to less fluorescence by removal of the fluorescent beads contained in the gel coating), and the portion of gel outside the microchannel wall remaining intact.

This exemplary system of microchannels and functionalized gel-coated surfaces (e.g., a microchannel device) was used to assess cell capture efficiency and cell release. Prior to use, the films were rehydrated with a buffer, and functionalized with avidin and an anti-human EpCAM antibody. Such preparation procedure is described, e.g., in Nagrath et al., “Isolation of rare circulating tumour cells in cancer patients by microchip technology,” Nature 450 (2007), pp. 1235-1239. H1650 cells were fluorescently labeled and spiked at a concentration of 1300 cells per mL into TBS. This cell suspension was then flowed through multiple microchannel devices as described herein, under shear stress conditions comparable to those typically present in a CTC chip. Effluent from the devices was then collected so that uncaptured cells could be enumerated.

Following cell capture from the flowing cell suspension by the functionalized surfaces, the devices were washed to remove unbound cells. This wash fluid was pooled with the effluent to collect substantially all uncaptured cells from the suspension. The microchannel devices were then either fixed with 1% paraformaldehyde, or the cells were released by dissolving the gel coating using a release buffer containing 5 mM EGTA.

The capture efficiency was calculated by counting the number of captured cells and dividing by the total number of cells passed through the device. The non-specific binding was assessed by omitting the antibody from the gel functionalization process, and repeating the procedure and measurements. Cell viability was also assessed by adding approximately 10,000 cells per mL to the release buffer (5 mM EGTA in RPMI) for 2 hours, and then determining for viability using a trypan blue exclusion technique and a live/dead fluorescence assay (Invitrogen, L3224). Viability of these cells was compared to that of cells kept in an RPMI solution without EGTA. Both tests indicated that cell viability was not affected by exposure to either solution.

Results of these procedures for capture efficiency, non-specific cell binding by the functionalized surfaces, and viability of captured cells are shown in Table 2. These results suggest that the exemplary functionalized coatings described herein are capable of exhibiting a relatively high capture efficiency and a relatively low capture rate for non-targeted cells. The results also indicate that a high percentage of the captured cells remain viable after they are released by dissolution of the gel coating.

TABLE 2
Capture and Release Effectiveness Data for Functionalized Alginate Gel

| | |
|---|---|
| Capture Efficiency (n = 3) | 70 ± 2.5% |
| Non-Specific Binding | 7.3% |
| Viability of Released Cells (n = 3) | Greater than 90% |
| | (Control sample—86%) |

Further features and aspects of the present invention are described in the following exemplary and non-limiting examples.

### EXAMPLES

### Example 1

### Preparation of a Cross-Linked Alginate Cell Capture System Using Calcium Chloride

This Example describes the preparation of a cell capture system using calcium chloride to cross-link a functionalized hydrogel comprising alginate. The alginate was attached to a surface using carbodiimide conjugation techniques to couple biotin hydrazide to the alginate. The exemplary cell capture system was prepared on a silicon CTC chip.

The surface of the CTC chip was grafted with an alginate priming layer and then spin coated with a biotin-alginate hydrogel and functionalized with an EpCAM antibody, as described herein and shown graphically in FIGS. 1A-1D.

The silicon-based CTC chips were first Piranha cleaned and then treated with an oxygen plasma treatment (2% O2, 50 W, 35 s). The surface was then immediately aminated by exposing it to a solution of 5% 3-Aminopropyltriethoxysilane, 90% ethanol, and 5% deionized water (having a pH of about 5) for 5 minutes. The CTC chips were rinsed in ethanol, nitrogen dried, and baked at a temperature of about 110° C. for 30 minutes. The CTC chips were then immersed in an Alginate/EDC/NHS solution (at a molar ratio of 1 uronic acid:3430 EDC:1715 Sulfo-NHS) with 1 mg/mL of alginate in a 50 mM MES buffer solution having a pH of about 6.5. The immersed chips were kept under vacuum for 45 minutes to reduce or eliminate trapped bubbles within the post structure of the CTC chip, and then incubated on a rocker for 14 hours followed by an hour rinse in deionized water. The CTC chips were then dried with nitrogen.

The biotinylated alginate was provided by preparing a 1% (w/v) solution of alginate (100 kD) in 50 mM MES buffer solution having a pH of about 6.5. Biotin hydrazide was mixed in for one hour (at a molar ratio of 1 uronic acid:0.2 biotin hydrazide) and EDC and Sulfo-NHS were then added (molar ratio: 1 uronic acid: 0.1 EDC: 0.05 Sulfo-NHS) and the solution was stirred for three hours. This material was then dialyzed (with a 10,000 MW cutoff limit) for 72 hours against deionized water at a ratio of 1 mL solution:60 mL water, which was changed every 24 hours. The functionalized alginate was then lyophilized and reconstituted at a 2% concentration in deionized water.

The gel coating of the CTC chip surface was formed using the following exemplary procedure. The 2% biotin-alginate solution described above was spun onto the galled substrate at 3000 RPM for 30 seconds and then air dried. To form crosslinked hydrogels, the films were then spray-crosslinked with a solution of 250 mM calcium chloride in TBS using an airbrush at 80 PSI pressure from a distance of about 8 inches.

The crosslinked gels were then rinsed in a solution of 2.5 mM calcium chloride in TBS (hereafter referred to as the “buffer solution”), and incubated with 10 μg/mL of streptavidin in the buffer solution for 45 minutes and rinsed again. The biotinylated EpCAM antibody was then incubated at a concentration of 10 μg/mL in the buffer solution for 45 minutes. The films were then rinsed with the buffer solution and nitrogen dried.

Unless otherwise specified, the biological samples tested below were processed under conditions typically used in conventional CTC sample processing. For example, blood samples were collected in lithium heparin vacutainers. The wash buffer and base buffer used for all other solutions was a 2.5 mM concentration of calcium chloride in TBS. The release solution used to dissolve the gel coatings was a solution of 5 mM EGTA in RPMI 1640, which was run through the CTC chips at a flow rate of about 10 mL/hour for 18 minutes, following the wash step.

### Example 2

### Effect of Variation of Biotins Per Alginate Chain on Antibody Binding

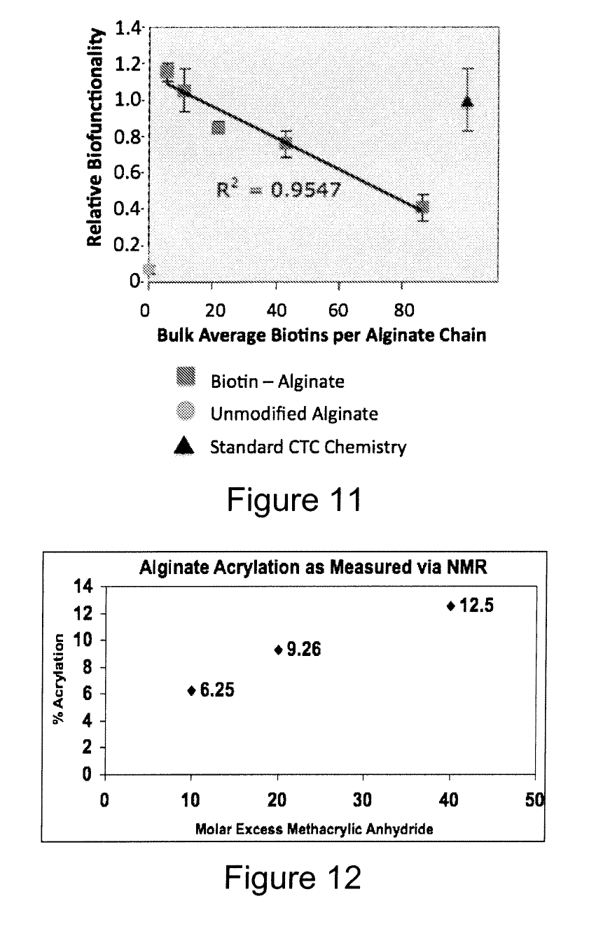

In this Example, we studied the relationship between the number of biotins included per alginate chain and the amount of biotinylated antibody that could be bound to the gel via a biotin-avidin sandwich style interaction in Example 1. We found that in fact low levels of biotinylation were much more successful at coupling antibody to the gel, as shown in FIG. 11. Furthermore, we found that this relationship appears to relate to the bulk average biotins per chain, as similar results are found by diluting highly functionalized (80 biotins per chain) alginate with unfunctionalized alginate, or by establishing uniform, low (5-10 biotins per chain) levels of biotinylation.

Carbodiimide conjugation techniques described herein can be used to couple biotin hydrazide to alginate, and a HABA assay can be used to quantify the degree of biotinylation. The relationship between biotins per alginate chain and the amount of biotinylated antibody that can be bound to the gel using a biotin-avidin sandwich technique was examined. Exemplary results of a study of this effect are shown in FIG. 11.

These observations suggest that low levels of biotinylation can be more effective for coupling an antibody to the gel than higher biotinylation levels. Similar effects were observed when diluting highly functionalized alginate (80 biotins per chain) with unfunctionalized alginate, and when establishing uniform, low levels of biotinylation (e.g., about 5-10 biotins per chain). Accordingly, the relationship shown in FIG. 11 appears to depend primarily on a bulk average number of biotins per chain. we found that the extent of biotinylation is important in incorporating biofunctionality, we found that chain length and/or polydispersity is important as well. We found that by using longer alginate chains (MW=220 kD vs MW=100 kD), we were able to achieve higher levels of biofunctionality. However, the 100 kD material had a wider polydispersity, which would result in a broad degree of biotinylation, and this may be the cause of this result.

### Example 3

### Preparation of an Acrylated Alginate Hydrogel Using Photocrosslinking

This example describes the preparation of photocrosslinked hydrogels comprising acrylated alginate. We also investigated preparing functionalized hydrogels by cross-linking acrylated alginate in the presence of a photoinitiator and UV light. This formed a covalent functionalized cross-linked hydrogels may be formed. The resulting functionalized hydrogel can be stable even in the presence of calcium chelators such as EDTA, a commonly used anticoagulant. Acrylation of the alginate can be performed by reacting the hydroxyl on alginate with an excess of methylacrylic anhydride, leaving the carboxyls available for biotinylation. FIG. 12 shows the percent acrylation observed as a function of the molar excess of methacrylic anhydride used. After successful acrylation, followed by adding photoinitiatior to these materials, we are able to form alginate gels that are stable for over 7 days in EDTA solutions.

We also formed an acryl modified alginate for photocrosslinking using a water soluble approach that is based on using N-(3-Aminopropyl)methacrylamide HCl. This methacryl-containing molecule (with an amine on one end) was mixed with biotin hydrazide and then both the N-(3-Aminopropyl)methacrylamide HCl and the biotin hydrazide are attached to the alginate using the same conjugation reaction to form an alginate polymer with a backbone that is ‘decorated’ with both biotin and methacryls at desired stoichiometric ratios. We made algiantes with a wide range of biotin densities (0-20%) and acryl densities (0%-75%), where the % refers to the starting ratio of the ligand to the number of free carboxyl groups on the alginates in solution. Furthermore, when up to 75% of the total number of available carboxyls were targeted, the efficiencies appeared to remain constant at approximately 55% for any combination of either ligand. The resulting material retained its ability to be calcium crosslinked, photocrosslinked in the presence of Irgacure 2959, and functionalized with avidin.

### Example 4

### Preparation of an Alginate Hydrogel Using Calcium Chloride and Photocrosslinking

This Example describes the formation of a cross-linked hydrogel material comprising acryl alginate using both calcium chloride and photocrosslinking to cross-link the acryl alginate hydrogel. We developed two approaches to form stable photocrosslinked alginate gels that remain gelled in the presence of calcium chelators.

In a first cross-linking approach, gels are formed by spincoating acryl alginate as previously described in Example 1, then spraying the gels with a solution of calcium chloride (at a concentration of 100-250 mM or higher). This instantly forms calcium crosslinked alginate gels. These gels are then be incubated in a calcium containing solution with a photoinitiator at appropriate concentrations (here, Irgacure 1959, 0.05-0.5%); following incubation, the gels are treated with UV light (30 sec to 10 minutes) to initiate free radical polymerization.

In the second cross-linking approach, photoinitiator is mixed with the acryl alginate prior to spincoating, and spun onto the substrate. The dry substrate is the treated with UV light to crosslink the films. These films then form stable gels when hydrated; this process eliminates the need for calcium crosslinking.

### Example 5

### Enzymatic Degradation of Cross-Linked Alginate Hydrogels

The photocrosslinked gels formed using the methods described in Example 4 were degradable using alginate lyase, a bacterial enzyme directed against the alginate backbone. The rapid gel degradation can be used to release captured cells.

In studying the degradation of our photocrosslinked gels, we learned that they are only degradable by lyase in a reasonable time frame (<5 min) if the crosslinking density is well controlled. Specifically, we found that acryl alginate gels photo-crosslinked in baths of 250 mM calcium chloride were unable to be reliably digested; however, those photo-crosslinked in 2.5 mM calcium chloride were able to be digested. We relate this to the density of covalent crosslinking, because the 250 mM solution pulls the alginate chains much closer together, enabling the free-radical propagation to reach more chains in a given path, compared to the case where the chains are further apart.

As we need to initially crosslink our gels with 250 mM calcium chloride to promote the instantaneous gelation, we studied the effects of varying the calcium concentration, and found that a two step approach worked best. In this case, the gels are initially crosslinked at 250 mM and then placed in successive baths of 2.5 mM calcium to ‘wash out’ the excess calcium. They remain stably crosslinked at 2.5 mM, and then may be photocrosslinked. The ensuing photocrosslinking forms a lower crosslink density as the chains have now relaxed in the lower concentration calcium bath, and may now be degraded with the addition of alginate lyase.

### Example 6

### Cell Capture and Release Using the Cell Capture System

The specific cell capture and release efficiency of the functionalized gel coatings described in Example 1 were tested using cultured cancer cells introduced into a blood sample that was processed using the exemplary cell capture system described in Example 1.

Fluorescently labeled H1650 lung cancer cell line cells were spiked into a whole blood sample at a concentration of 5000 cells/mL. The H1650 non-small lung cancer cell line cells were obtained from ATCC. These cells were cultured in RPMI 1640 medium with 10% fetal bovine serum and 1% Penicillin-Streptomycin at 37° C., 5% CO2, and were split when flasks were at 70-80% confluence using 0.05% trypsin-EDTA. The cells were labeled by treating them with 10 μM cell tracker orange (Invitrogen Corp.).

The blood and cells were mixed, and then processed through three CTC chips in parallel procedures for comparison purposes. The CTC chips used were: (a) a standard CTC chip used to quantify a baseline cell capture behavior; (b) an alginate-coated CTC chip that was fixed following a wash step to evaluate capture performance on the alginate coating; and (c) an alginate-coated CTC chip from which the captured cells were released following the wash step using a release buffer solution to dissolve the alginate coating as described herein. This latter CTC chip was fixed and imaged after flowing 6 mL of the release buffer solution flowed through the chip to evaluate the effectiveness of the release process. All three CTC chips were stained with a DAPI nuclear stain and imaged for both the specific fluorescent stain and the DAPI stain on a scanning microscope. The entire capture area on each CTC chip was imaged to assess cell capture and release performance.

After cells were released from the alginate coated chip, about 10% of the estimated number of captured cells remained on the chip. The solution containing released cells included approximately 3000 cells per mL of blood processed, as counted under fluorescence using a hemocytometer. Together, these data indicate that the release efficiency is about 90%.

Results of this analysis are presented in Table 3 below. The alginate-coated chip exhibited a capture efficiency that is comparable to the control (uncoated) CTC chip. This exemplary study suggests that that the capture efficiency from whole blood of the alginate coated chip was at least comparable with that of a standard CTC chip, and the alginate system was able to release 90% of the captured cells. Thus, the addition of a sacrificial alginate hydrogel layer does not appear to affect the interactions between the cell surface and the capture antibody or significantly change the fluidic behavior of the sample on the CTC chip.

TABLE 3
Comparison of Capture and Release Efficiency

| | Cells captured per mL of | Capture | Release |
|---|---|---|---|
| | Whole Blood Processed | Efficiency | Efficiency |
| Control Chip | 3291 | 66% | N/A |
| Alginate Chip | 3609 | 72% | 90% |

### Example 7

### Patient CTC Capture and Release Analysis

To assess specific cell capture and release capabilities of the functionalized gel coatings described in Example 1, patient CTCs were captured, released, and immunostained for specific cancer markers using a CTC chip comprising the Cell Capture System in Example 1, coated with a functionalized gel that was prepared as described in Example 1. Blood samples were obtained from a prostate cancer patient with known metastases and high CTC counts. Blood was collected in a lithium heparin vacutainer. CTCs from this sample were selectively captured using an alginate coated CTC chip prepared as described herein. The CTCs were then released by dissolving the alginate coating using a release buffer.

The CTCs were imaged immediately after being released from the CTC chip. An image of the released cells is shown in FIG. 10. The granulated cells shown in FIG. 10 (a few of which are indicated by black arrows) are CTCs that were isolated from the blood sample and then released in accordance with exemplary embodiments of the present invention. The scale marker in the lower right corner of FIG. 10 is 10 μm in length.

The released cells were then incubated in RPMI medium in a multiwell culture plate overnight to allow the CTCs to attach to the surface. The next day, the well was gently rinsed to remove any unbound cells (presumably the RBCs and leukocytes) and then fixed and immunostained for a DAPI nuclear stain and one of either a pan-cytokeratin or prostate specific antigen (PSA).

Pan-cytokeratin staining was conducted as follows (including a wash step with PBS between each step): the sample was fixed in 4% paraformaldehyde for 1 hour, permeabilized with 0.2% Triton-X for 45 minutes, and stained with a FITC conjugated mouse pan-cytokeratin antibody (Abeam ab11212, Cambridge, Mass.) used at a concentration of 37.5 μg/mL for one hour and a DAPI nuclear stain (1:1000) for 20 minutes.

PSA staining was conducted as follows (including a wash step with PBS containing 10 mM glycine between each step): the sample was fixed with 4% paraformaldehyde for 30 minutes, permeabilized with 1% NP40, and then blocked with 3% BSA and 2% goat serum for 30 minutes. The primary polycolonal rabbit anti-human PSA antibody (Dako A0562) was then incubated at a concentration of 3 μg/mL for one hour. The Alexa Fluor 488 labeled goat antirabbit secondary antibody (A11008, Invitrogen, Carlsbad, Calif.) was incubated for one hour at concentration of 2 μg/mL, followed by the DAPI stain (1:1000) for 5 minutes.

Both staining techniques confirmed that the captured and released cells were CTCs. These results suggest that embodiments of the present invention that include a functionalized hydrogel coating can be quickly deployed for field use and other clinical applications to achieve efficient detection of specific cancer markers from small blood samples.

### Example 8

### Comparing Dissolution vs. Delamination as Functions of Shear Stress

This Example describes how applied shear stress impacts the mechanisms by which an alginate hydrogel is released from the underlying substrate in cases where there is a thin alginate coating (˜1 um). Other parameters are thought to govern when the hydrogel is a bulk material rather than a coating applied to a substrate.

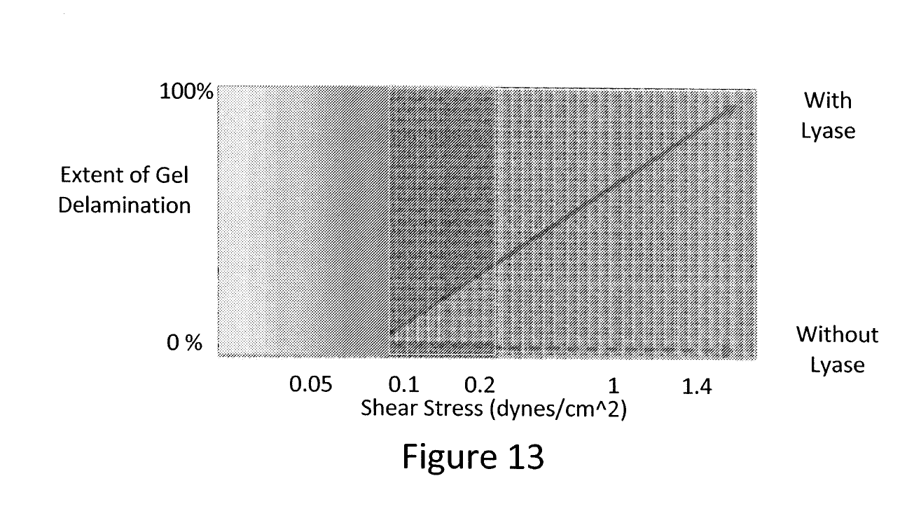

Referring to FIG. 13, varying shear stress were applied to microfluidics devices with cells captured by a functionalized alginate coating. The experiment was conducted using the techniques and tools previously outlined, and is a mix of the above examples. The material used was an acrylated, biotinylated alginate that was spun coat onto a glass slide. The gel was formed by first calcium crosslinking with a 250 mM airbrushed spray, then soaking in 2.5 mM to wash out the excess calcium as previously described. Irgacure 2959 was then added at 0.05% and the film was exposed to UV for 30 seconds. A microfluidic channel was then clamped on top as shown previously. The cell experiments were based on the cell line experiments also previously discussed here. Finally, the entire system was visualized using fluorescent microscopy where the cells were labeled with cell tracker orange and the gels was detected using the impregnated 50 nm beads. In a first flow regime at shear stresses below ˜0.1 dynes/cm², full dissolution of the gel was observed upon application of lyase, resulting in clean single cell release. In a second flow regime at shear stresses between 0.1 and 0.2 dynes/cm², mixed dissolution and delamination of the alginate coating with small fragments of the gel coming off of the surface was observed. Some single cell release was achieved, particularly when the spatial frequency of cells was low. In the third flow regime at shear stresses above 0.2 dynes/cm², delamination dominated. The cell release became less reliable and, in some instances, the film tore around individual cells, leaving the cells (with a patch of underlying gel) still adhering to the substrate. At shear stresses above 1.4 dynes/cm², the dominant observed effect of delamination approaches 100% of the release. It was also observed that even at high shear stresses (e.g., 20 dynes/cm²) delamination did not occur in the absence of lyase. The foregoing merely illustrates the principles of the invention. Various modifications and alterations to the described embodiments will be apparent to those skilled in the art in view of the teachings herein.

For example, dissolvable material such as an alginate hydrogel can be incorporated into the herringbone device described in Int. Pat. App. Pub. No. WO 2010/036912(A2) using different fabrication methods other than those described above. In some of these methods, the alginate is patterned on a glass slide to fit below the herringbone structure before a PDMS (patterned elastomer) piece is bonded on top of the glass slide. Other methods such as spincoating, and/or alternate deposition techniques such as spotting, or spraying, can be used to apply the alginate onto the structured geometry of the PDMS piece.

In some embodiments, an entire cell capture device can be formed (e.g., molded) out of a dissolvable material rather than having a dissolvable material applied to an underlying structure of the cell capture device.

It will thus be appreciated that those skilled in the art will be able to devise numerous techniques which, although not explicitly described herein, embody the principles of the invention and are thus within the spirit and scope of the invention. All patents and publications cited herein are incorporated herein by reference in their entireties.

1-58. (canceled)

59. A method of selectively capturing and releasing one or more target cells from a sample, the method comprising
(a) obtaining a functionalized hydrogel composition, wherein the hydrogel composition comprises
(i) a hydrogel that is at least partially covalently crosslinked, and
(ii) one or more cell-binding moieties that selectively bind to the target cells;
(b) contacting a sample to the functionalized hydrogel composition under conditions effective to enable the one or more cell-binding moieties to bind to target cells in the sample; and
(c) dissolving at least a portion of the functionalized hydrogel composition to release the target cells from the substrate.

60. The method of claim 59, wherein the functionalized hydrogel composition comprises a photoinitiator and is at least partially photocrosslinked.

61. The method of claim 59, wherein the functionalized hydrogel composition is bound to a substrate.

62. The method of claim 59, further comprising removing unbound cells from the functionalized hydrogel composition before dissolving step (c).

63. The method of claim 59, further comprising detecting the released target cells.

64. The method of claim 63, wherein detecting comprises staining of the target cells.

65. The method of claim 59, wherein the one or more target cells are viable living cells.

66. The method of claim 65, further comprising maintaining the released target cells under conditions effective to culture and grow the viable living target cells.

67. The method of claim 59, wherein the hydrogel comprises an alginate.

68. The method of claim 59, wherein the hydrogel that is at least partially covalently crosslinked is further partially ionically crosslinked.

69. The method of claim 59, wherein dissolving at least a portion of the functionalized hydrogel composition comprises contacting the functionalized hydrogel composition with an enzyme.

70. The method of claim 69, wherein the enzyme comprises a lyase that at least partially dissolves the hydrogel composition.

71. The method of claim 70, wherein the hydrogel comprises alginate and the lyase comprises alginate lyase.

72. The method of claim 59, wherein the sample is whole blood.

73. The method of claim 59, wherein the cell-binding moieties selectively bind to leukocytes, CD4+ T-cells, or circulating tumor cells (CTCs).

74. The method of claim 59, wherein the cell-binding moieties comprise antibodies.

75. The method of claim 59, wherein the cell-binding moieties comprise one or more binding molecules selected from the group consisting of: biotin, avidin, aptamers, polynucleotides, and selectins.

76. The method of claim 59, further comprising
growing the released target cells in cell culture media;
characterizing the cells with one or more stains;
characterizing the cells with identification of the cellular DNA or RNA;
measuring the division rate of the cells; and
transforming the cell with external chemicals or biomolecules.

77. The method of claim 59, wherein the cells are circulating tumor cells (CTC) and the one or more cell-binding moieties comprise an anti-EpCAM antibody.

78. A method of making a cell capture surface, the method comprising:
depositing a hydrogel material onto a silicon-containing surface to form a layer of the hydrogel material less than 10 microns thick on the surface, the hydrogel comprising a cross-linkable polysaccharide;
cross-linking the hydrogel material on the surface;
contacting the cross-linked hydrogel material with a cell-binding moiety under conditions effective to bind the cell-binding moiety to the cross-linked hydrogel material, thereby forming the cell capture surface.

79. A target cell capture device comprising:
a solid substrate; and
a cross-linked functionalized hydrogel composition bound to the substrate, wherein the functionalized hydrogel composition comprises (i) a hydrogel that is at least partially covalently crosslinked, and (ii) one or more cell-binding moieties that selectively bind to the target cell.

80. The target cell capture device of claim 79, wherein the hydrogel is a polysaccharide.

81. The target cell capture device of claim 79, further comprising a primer material covalently bound to the solid substrate, wherein the functionalized hydrogel composition is covalently bound to the primer material.

82. The cell capture device of claim 79, wherein the solid substrate comprises a silica-containing material.

82. The cell capture device of claim 79, wherein the polysaccharide comprises biotinylated alginate.

83. The cell capture of device of claim 79, wherein the cell-binding moiety comprises an anti-EpCAM antibody.
